(19) United States
(12) Patent Application Publication
(10) Pub. No.: US 2024/0197520 A1
(43) Pub. Date: Jun. 20, 2024

(54) POSTPARTUM HEMORRHAGE MONITORING DEVICE AND METHODS

(71) Applicant: CooperSurgical, Inc., Trumbull, CT (US)

(72) Inventors: Andrew P. Isch, Lafayette, IN (US); Matthew R. Jones, Indianapolis, IN (US); Eugene Ferrari, Lafayette, IN (US); Willie C. McRoy, JR., West Lafayette, IN (US); Ashley Molnar, Coal City, IL (US); Christian Wagner, West Lafayette, IN (US); Kevin L. Delaney, Bloomington, IN (US)

(21) Appl. No.: 18/539,592

(22) Filed: Dec. 14, 2023

Related U.S. Application Data
(60) Provisional application No. 63/387,449, filed on Dec. 14, 2022.

(51) Int. Cl. A61F 5/455; A61F 5/44

(52) U.S. Cl. CPC A61F 5/4553; A61F 5/4404

(57) ABSTRACT

A post-partum hemorrhage monitoring system has a fluid collection funnel having a opening configured to receive post-partum hemorrhaging, an open distal end, a base, an interior configured to receive postpartum hemorrhage, a lip extending exteriorly from the open proximal end, an anterior surface configured to be placed adjacent an anterior vaginal wall of a patient, a posterior surface configured to be placed adjacent a posterior vaginal wall of a patient and a stem having a lumen extending from an open distal end of the funnel. The anterior surface tapers from the lip to and through the base, the posterior surface has substantially no taper from the lip to and through the base to provide a flattened flow path, and the stem is offset from the geometric center of the fluid collection funnel.

### CROSS REFERENCE TO RELATED APPLICATION

This application claims priority to U.S. Provisional Patent Application No. 63/387,449, filed on Dec. 14, 2022, pursuant to 35 USC § 119. The content of this provisional application is incorporated herein by reference in its entirety.

### TECHNICAL FIELD

The present disclosure relates to devices for collecting and accurately measuring postpartum hemorrhaging.

### BACKGROUND

The American College of Obstetricians and Gynecologists (ACOG) defines postpartum hemorrhaging (PPH) as cumulative blood loss greater than or equal to 1,000 ml, or blood loss accompanied by signs or symptoms of hypovolemia within 24 hours after the birth process. PPH is the leading cause of maternal mortality, affecting 5% of women giving birth worldwide. Late recognition and delayed management of PPH have been identified as the main contributing factors to maternal death. Current methods for PPH detection include visual estimation of the blood-soaked mat, gauze, towels, and pads; referencing a chart with images and estimated volumes; weighing pads before and after use; and under buttocks drapes that collect fluid.

Visual estimation is inaccurate and unreliable as it does not improve with specialty, age, or clinical experience. Weighing pads before and after use requires waiting until the pads are blood-soaked, leading to delays in blood loss monitoring. Under buttocks drapes are prone to accidents and low in both quality and accuracy. These methods lead to inaccuracies and late identification of PPH, hindering timely management of PPH. This presents a need for a quantitative, cumulative blood loss monitoring solution that is quick to implement.

### SUMMARY

The apparatuses and methods of the present disclosure provide a blood collection device for placement in the female anatomy after giving birth to passively drain hemorrhaging blood into a collection and measurement vessel. The insertable portion of the blood collection system is connected to a drainage tube which in turn is connected to a collection vessel/receptacle. The system provides rapid identification and accurate measurement of post-partum blood loss after delivery. By more accurately determining when blood loss has crossed the critical threshold, the disclosed solutions may provide early detection for PPH leading to improved patient outcomes.

The postpartum hemorrhage monitoring system includes a fluid collection funnel having a symmetrically circular opening configured to receive post-partum hemorrhaging, an open distal end, a base, an interior configured to receive postpartum hemorrhage, a symmetrically circular lip extending exteriorly from the symmetrically circular open proximal end, an anterior surface configured to be placed adjacent an anterior vaginal wall of a patient, a posterior surface configured to be placed adjacent a posterior vaginal wall of a patient, an open distal end, and a stem having a lumen extending from the open distal end, wherein the anterior surface tapers from the symmetrically circular lip to and through the base, wherein the posterior surface has substantially no taper from the symmetrically circular lip to and through the base to provide a flattened flow path, and wherein the stem is offset from the geometric center of the fluid collection funnel.

The fluid collection funnel may also include a pressure point or region adjacent the lip that is designed and configured to facilitate folding of the fluid collection funnel, which may be of a different durometer, thickness, and or material than the rest of the funnel. The system may further include drainage tubing extending from the open end of the stem to a free-standing, self-supporting, expandable, fluid collection receptacle. The collection receptacle may be configured to be collapsed, folded and/or compressed for packing within packaging or storing, and self-expanding upon release from the package. The lip of the fluid collection funnel may be thinner at the top (anterior) of the lip to facilitate folding.

The apparatuses and methods further provide a fluid collection system that includes a first fluid collection funnel having an elongate stem/drainage tubing that has a second fluid collection funnel at the opposite end of the stem. The first fluid collection funnel of the doubled-sided device has a small profile funnel at one end and a larger profile funnel at the other end. The cups have identical features or they have different features. The collection funnels inner geometries on both sides of the drainage tubing will be fitted with a barbed style connector or like. The drainage tubing will have a similar connector designed to mate with the connector at each funnel.

One general aspect may include a fluid collection device of a postpartum hemorrhage monitoring system. The fluid collection device may include a funnel body having an open proximal end, an interior configured for receiving a fluid, a rim extending exteriorly from the open proximal end, an anterior outer surface configured for placement adjacent an anterior vaginal wall of a patient, a posterior outer surface configured for placement adjacent a posterior vaginal wall of the patient, and an open distal end.

Another general aspect may include a fluid collection device for a postpartum hemorrhage monitoring system. The fluid collection device may include a funnel body having an open proximal end, an interior configured to receive postpartum hemorrhage, a symmetrically circular rim extending exteriorly from the circular open proximal, an anterior outer surface configured for placement adjacent an anterior vaginal wall of a patient, a posterior outer surface configured for placement adjacent a posterior vaginal wall of a patient, an open distal end, and a pressure point to facilitate folding the funnel for placement with a patient. The device may include a stem having an open proximal stem end, an open distal stem end, and a lumen extending therebetween. The stem may extend from the open distal end of the funnel body and may be configured for receiving fluid from the interior of the funnel body. The anterior outer surface may taper toward the proximal stem end. The posterior outer surface may be substantially flush with the stem. The stem may be offset from a geometric center of the funnel body to facilitate movement of fluid into and through the stem and minimize accumulation of fluid within the funnel body when the patient is in a supine position.

In another general aspect, the postpartum hemorrhage monitoring system may include a drainage tube and a collapsible collection bag configured for coupling to the drainage tube. The system may include a fluid collection funnel configured for coupling to the drainage tube. The fluid collection funnel may include an open proximal end, an interior configured to receive postpartum hemorrhage, a rim extending exteriorly from the open proximal end, an anterior outer surface configured for placement adjacent an anterior vaginal wall of a patient, a posterior outer surface configured for placement adjacent a posterior vaginal wall of a patient, and an open distal end. The system may include a stem having an open proximal stem end, an open distal stem end, and a lumen extending therebetween. The stem may extend from the open distal end of the fluid collection funnel and may be configured for receiving fluid from the interior of the fluid collection funnel. A pressure point may be disposed adjacent the rim to facilitate folding of the rim and the fluid collection funnel for placement within a patient. The anterior surface may taper to the proximal stem end and the posterior surface may be flush with the stem such that the stem is substantially rearward of a geometric center of the fluid collection funnel to facilitate movement of fluid into and through the stem and minimize accumulation of the fluid within the fluid collection funnel when the patient is in a supine position.

Implementations may include one or more of the following aspects.

In accordance with certain aspects of the present disclosure, the device may include a stem having an open proximal stem end, an open distal stem end, and a lumen extending therebetween.

In some aspects, the stem may extend from the open distal end of the funnel body and may be configured for receiving fluid from the interior of the funnel body.

In accordance with some aspects, the anterior surface may taper to the proximal stem end and the posterior outer surface may be flush with the stem such that the stem is rearward of a geometric center of the funnel body to facilitate movement of fluid into and through the stem and minimize accumulation of fluid within the funnel body when the patient is in a supine position.

In some aspects, the stem may be entirely rearward of the geometric center of the funnel body.

In some aspects, a pressure point may be disposed adjacent the rim to facilitate folding of the rim and the funnel body for placement within a patient.

In some aspects, the pressure point may be disposed in an area of the funnel body having a durometer less than a durometer of a remainder of the funnel body.

In some aspects, the pressure point may be disposed adjacent the rim.

In some aspects, the pressure point may include an indentation.

In some aspects, the pressure point may be an area of the funnel body that is thinner than a remainder of the funnel body.

In some aspects, the device may include a second funnel body extending from the open distal stem end.

In some aspects, the second funnel body may be smaller than the funnel body.

In some aspects, the drainage tube may be configured for coupling to the open proximal end of the stem, and the collapsible collection bag may be configured for receiving fluid from the fluid collection funnel by way of the drainage tube.

In some aspects, the collapsible collection bag may have a collapsed configuration and an expanded configuration.

In some aspects, the collapsible collection bag may be freestanding in the expanded configuration.

In some aspects, the collapsible collection bag may be self-expanding upon release from the collapsed configuration.

### BRIEF DESCRIPTION OF THE DRAWINGS

FIG. 1 shows a blood loss monitoring system/kit;

FIG. 2 shows a perspective view of a fluid collection funnel having a circular contour;

FIG. 3 shows another perspective view of the fluid collection funnel of FIG. 2;

FIG. 4 shows a perspective view of a fluid collection funnel having a semicircular contour;

FIG. 5 shows a perspective view of the semicircular fluid collection funnel of FIG. 4;

FIG. 6 shows another example of a semicircular fluid collection funnel having an extended portion;

FIG. 7 shows a perspective view of the semicircular fluid collection funnel of FIG. 6;

FIG. 8 shows the circular fluid collection funnel of FIGS. 2 and 3 having a shorter stem;

FIG. 9 shows the semicircular fluid collection funnel of FIGS. 4 and 5 having a shorter stem;

FIG. 10 shows the semicircular fluid collection funnel of FIGS. 6 and 7 having a shorter stem;

FIGS. 11A and 11B show views of a circular fluid collection funnel folded for insertion;

FIGS. 12A and 12B show views of a semicircular fluid collection funnel folded for insertion;

FIGS. 13A and 13B show views of a semicircular fluid collection funnel folded for insertion;

FIG. 14A shows a perspective view of another example of a fluid collection funnel;

FIG. 14B shows a side view of the fluid collection funnel of FIG. 14A;

FIG. 14C shows an end view of the fluid collection funnel of FIG. 14B;

FIG. 15A shows a perspective view of another example of a fluid collection funnel;

FIG. 15B shows a side view of the fluid collection funnel of FIG. 15A;

FIG. 16A shows a perspective view of another example of a fluid collection funnel;

FIG. 16B shows a side view of the fluid collection funnel of FIG. 16A;

FIG. 17 shows a front view of a fluid collection;

FIG. 18 shows a perspective view of the cylindrical fluid collection receptacle of FIG. 17;

FIG. 19 shows a perspective view of a rectangular fluid collection receptacle;

FIG. 20 shows a front view of the rectangular fluid collection receptacle of FIG. 19;

FIG. 21 shows a receptacle with example graduation markings;

FIG. 22 shows a side profile of the rectangular fluid collection bag of FIGS. 19 and 20 folded in packaging and deployed for use;

FIG. 23 shows patient anatomy and a system for monitoring post-partum hemorrhaging in the patient prior to insertion of a fluid collection funnel;

FIG. 24 shows patient anatomy and a system for monitoring post-partum hemorrhaging in the patient after insertion of a fluid collection funnel;

FIG. 25 shows a different example of a blood-loss monitoring system assembled in accordance with the teachings of the present disclosure;

FIG. 26 shows an example of a double-sided fluid collection device; and

FIG. 27 shows another example of a double-sided fluid collection device.

### DETAILED DESCRIPTION

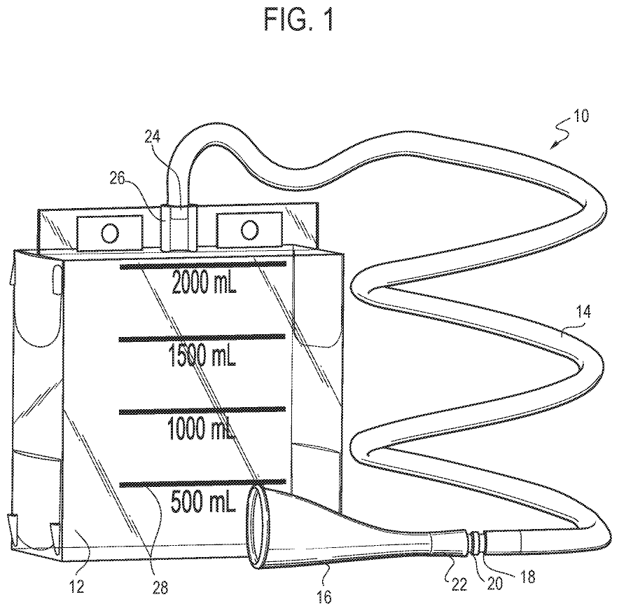

The present disclosure provides a blood loss collection system that provides accurate measurement and timely identified PPH. FIG. 1 shows a blood loss collection system 10. The system 10 includes a free-standing, self-supporting, and expandable fluid collection receptacle 12, drainage tubing 14, and a fluid collection funnel device 16. A first end 18 of the fluid connection end of the tubing 14 is sealingly connected to a drainage end 20 of a stem 22 of the fluid collection funnel device 16. A second end 24 of the drainage tubing 14 is sealingly connected to a port 26 of the collection receptacle 12. The collection receptacle 12 includes markings 28 to indicate the collected volume of blood. The drainage tubing 14 extends from the collection receptacle 12 to the stem 22 of the fluid connection funnel device 16. The stem 22 of the funnel device 16 connects to the first end 18 of the tubing 14 by friction-fit, and in some cases, by a conical connector. As will be described, the fluid collection receptacle 12 is designed and configured to be free-standing, expandable, and compressible for packaging.

In FIG. 3, a funnel body 40 of a fluid collection funnel device 16 would be placed intra-vaginally between the introitus and the cervix, and behind the pubic bone, following the delivery of the placenta after birth. The fluid collection funnel device 16 will funnel blood through the drainage tubing 14 into the fluid collection receptacle 12 secured at the side of the bed. This solution provides both quantitative and cumulative blood loss measurement and PPH can be identified more accurately and rapidly than current solutions. The fluid collection receptacle 12 will be labeled at increments of 50 or 100 ml to allow physicians to actively monitor blood loss and respond when the PPH threshold has been crossed. Additionally, the fluid collection receptacle 12 can be sealed and secured at the bedside making this solution less prone to accidents or spilling. Additionally, it can be placed on the floor next to the patient bed-side as it is free-standing.

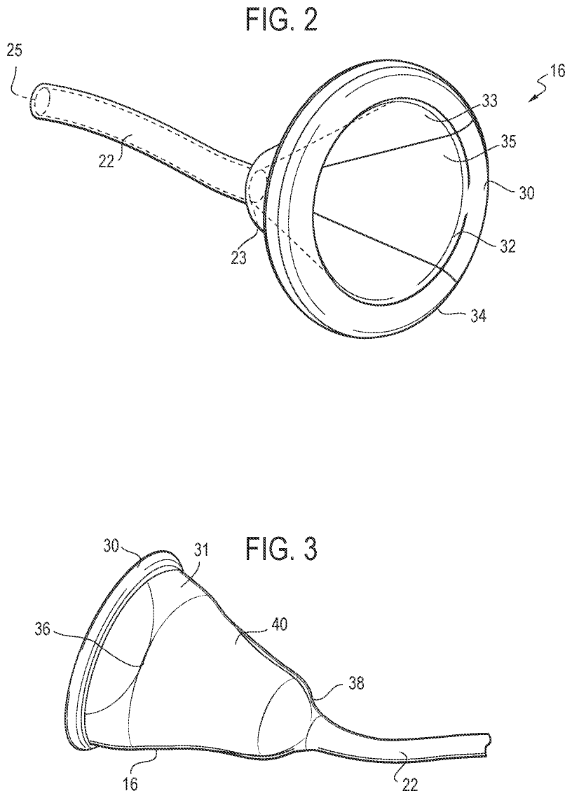

In one example of FIGS. 2 and 3, a fluid collection funnel device 16 has a circular contour. The fluid collection funnel device 16 is the part of the system that is inserted into and anchored in a patient's vagina postpartum and receives blood and other fluid for collection and measurement in the fluid collection receptacle 12. A protruding lip 30 provides a radial stiffness to the funnel device 16, and creates a firm anchoring feature against the vaginal walls. In this example the interior-most edge 32 of the protruding lip 30 is concentric with its exterior-most edge 34. In one example, the protruding lip 30 may slope inwardly from the exterior-most edge 34 to the interior-most edge 32. In another example, the protruding lip 30 may slope inwardly from the interior-most edge 32 to the exterior-most edge 34. The protruding lip 30 extends outwardly and uniformly from the exterior sidewall 31 of the fluid collection funnel device 16 at the opening 33, and is flush with the interior side wall 35 of the fluid collection funnel device 16 so as not to extend into the interior of the funnel body 40. This keeps the interior surface of the fluid collection funnel 16 unobstructed and unimpeded from any structure that would interfere with the laminar flow of fluid through and out of the funnel body 40. A smooth interior surface also avoids pooling and clotting of blood, which can lead to inaccurate measurement or obstruction of the fluid flow.

In FIG. 3, an area adjacent 36 to and extending distally from the protruding lip 30 has a thinner section 36 that allows the fluid collection funnel device 16 to easily fold on itself for insertion. This thinner section 36 may be located in the upper half of a funnel body 40 of the fluid collection funnel device 16, and in some examples in an upper third of the funnel body 40 of the fluid collection funnel device 16. The thinner section 36 gradually thickens both circumferentially and axially along the fluid collection funnel device 16. The fluid collection funnel device 16 has its maximum thickness 90° circumferentially from the thinner section 36 and at the base 38 of the funnel body 40 of the fluid collection funnel device 16. The funnel body 40 of the fluid collection funnel device 16 is contoured on one side while narrowing to the base 38 to provide some additional anchoring as the vaginal walls collapse.

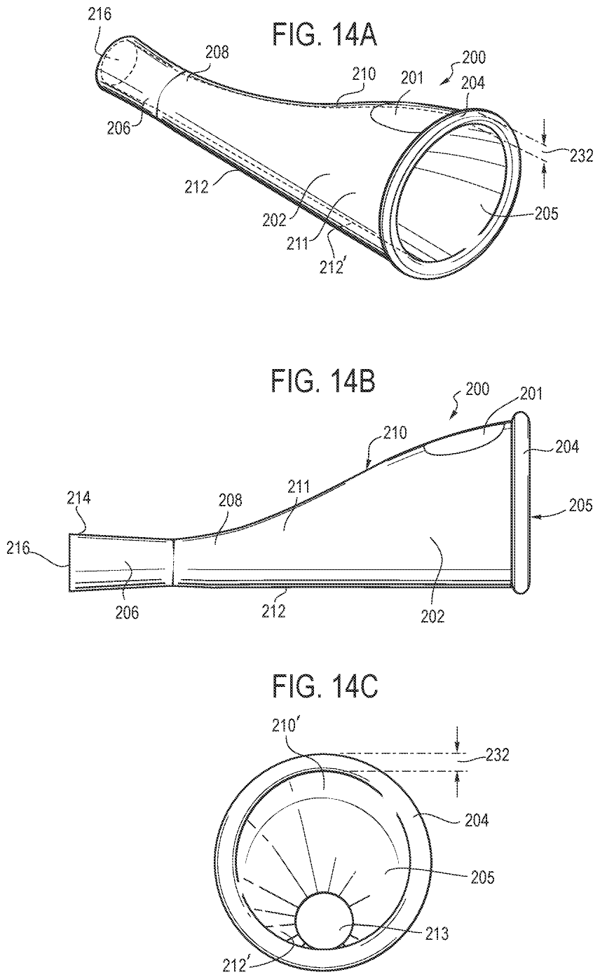

A portion (shown as element 232 in FIGS. 14A-C and 15A) of the protruding lip 30 at the top of the protruding lip 30 and adjacent the thinner section 36 also may be thinner than the rest of the protruding lip 30 to further facilitate folding of the fluid collection funnel device 16. In this configuration, both the protruding lip 30 and the opening 33 are symmetrically circular. In place of or in addition to the thinner section, the fluid collection funnel 16 may also include a pressure point or region (shown as element 201 in FIGS. 14A-B and 15A-B) on the anterior surface of the fluid collection funnel device 16 adjacent the protruding lip 30. The pressure point or region may be a region of the side wall 35 that is of thinner material, of a different durometer, and/or of a different material. The pressure point or region is located in the upper half, and in some examples an upper third, of the funnel body 40 of the fluid collection funnel device 16. In other examples, the protruding lip 30 on the anterior side may be thinner than the rest of the protruding lip 30 to facilitate folding of the fluid collection funnel device 16.

As shown in FIG. 2, the stem 22 extends between an open end 23 of the base 38 and an open end 25 of the stem 22, and is designed and configured to be attached to the drainage tubing 14 for transporting fluid to a fluid collection receptacle 12 for monitoring and measurement. In some examples, the fluid collection funnel 16 is constructed from soft, medical-grade silicone and is free from phthalate, latex, dioxin, & BPA. The durometer may range from 40A to 70A and, in some examples, is from about 50A to 60A. The thinner section and/or pressure point 201 or region may have of a lower durometer and in the range of about 20A to 40A.

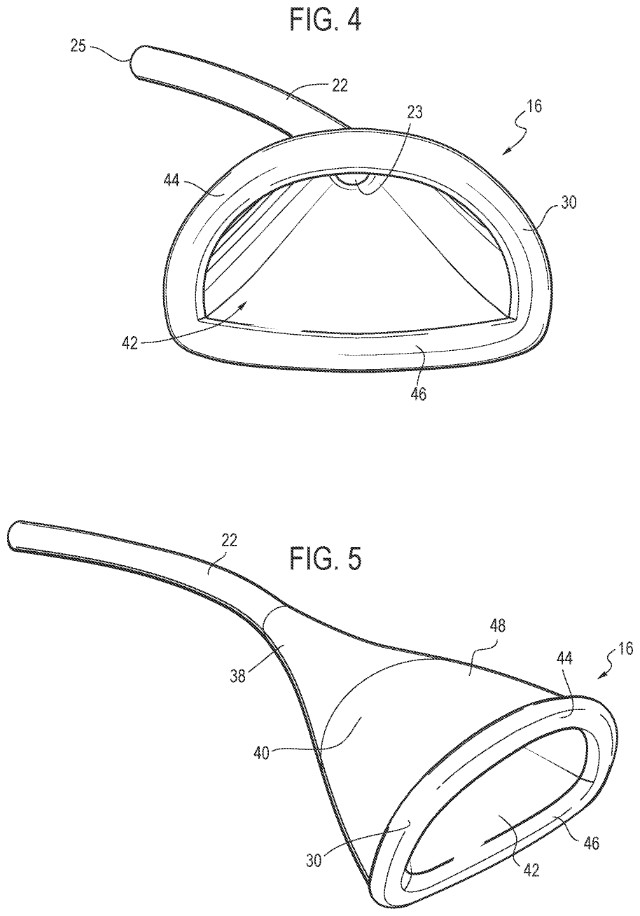

FIGS. 4 and 5 show a fluid collection funnel device 16 having a noncircular contour. As shown, the device 16 includes a funnel body 40 and a stem 22. A mouth 42 of the fluid collection funnel device 16 has a semicircular contour 44 with a shallow arc contour 46. The different contour shapes 44, 46 may increase the ergonomics and comfort of the device 16. The protruding lip 30 creates a firm anchoring feature with which the fluid collection funnel device 16 can press against the vaginal walls. A wall portion 48 of the fluid collection funnel device 16 adjacent and extending from the protruding lip 30 may be thinner that allows the device to easily fold on itself for insertion. The thinner section gradually thickens both circumferentially and axially along the semicircular portion 44 of the device 16, thereby achieving its maximum thickness at the intersection of the semicircular contour 44 and shallow arc contour 46 as well as at the base 38 of the funnel body 40. As shown, the funnel body 40 is contoured while narrowing to the base 38 to provide some additional anchoring as the vaginal walls collapse. As described above, the protruding lip 30 adjacent the thinner section also may be thinner than the rest of the protruding lip 30 to further facilitate folding of the fluid collection funnel device 16. In place of or in addition to the thinner section, the fluid collection funnel device 16 may also include a pressure point or region (201, as shown in FIGS. 14A-B and 15A-B) on the anterior surface of the fluid collection funnel 16 adjacent the protruding lip 30. Each of the features described with regard to FIGS. 2 and 3 and FIGS. 14 and 15 may also be included in this example.

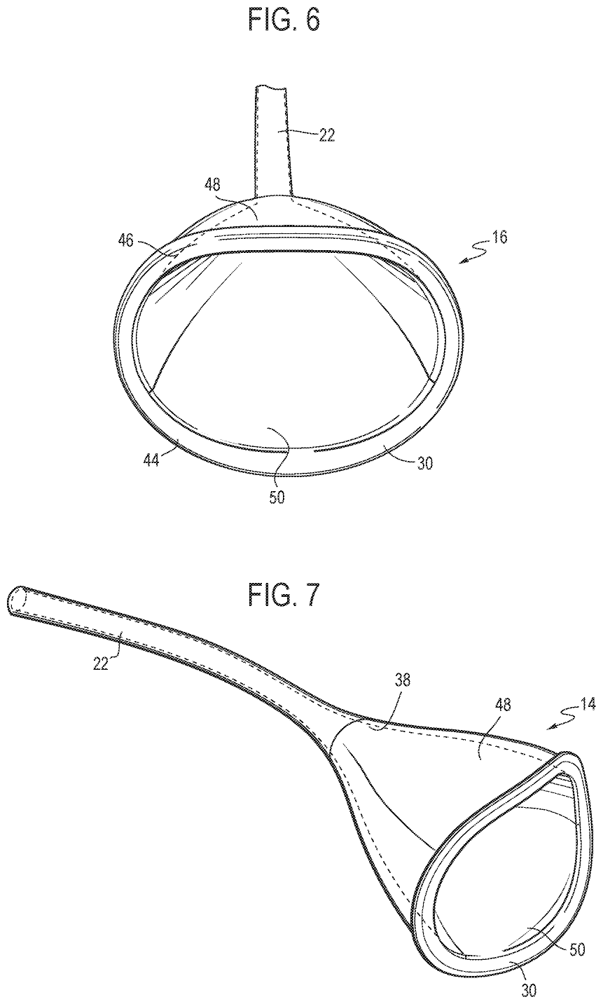

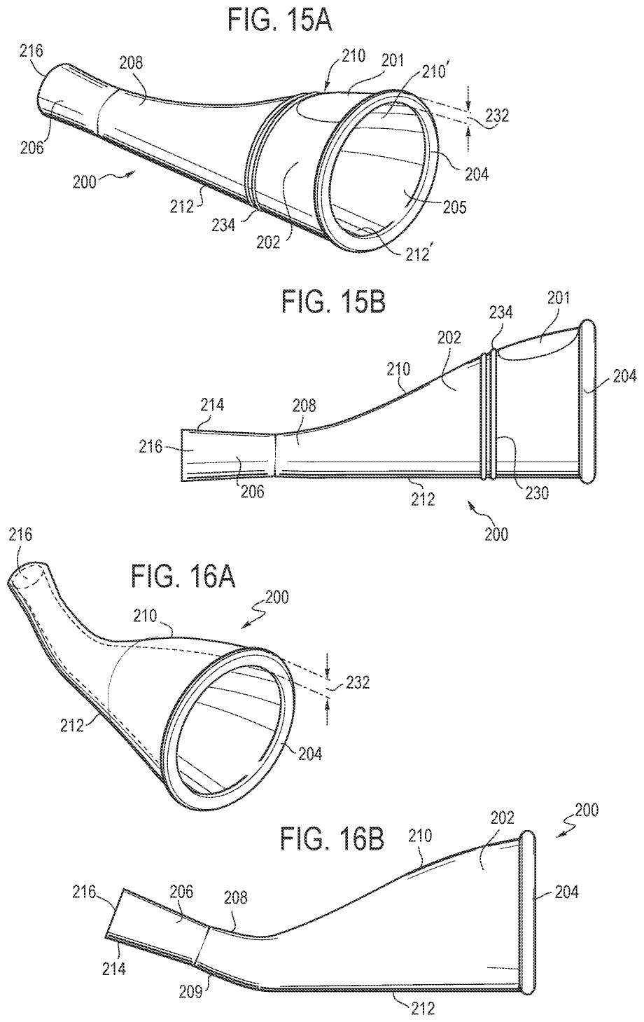

FIGS. 6 and 7 show another fluid collection funnel device 16 also having a noncircular contour, but with an extended portion 50. The fluid collection funnel device 16 in this example has a semicircular contour 44 and a shallow arc contour 46. The different contour shapes 44, 46 may increase the ergonomics and comfort of the device 16. A protruding lip 30 creates a firm anchoring feature with which the fluid collection funnel device 16 can press against the vaginal walls. The shallow arc contour 46 also forms a distal extension 50 designed to follow the angle of the vaginal canal relative to the uterus. As described above, the wall portion 48 of the fluid collection funnel 16 adjacent the protruding lip 30 may be thinner to allow the device 16 to easily fold on itself for insertion. The thinner section gradually thickens both circumferentially and axially along the semicircular portion 44 of the device 16 and the fluid collection funnel device 16 achieves its maximum thickness at the intersection of the semicircular contour 44 and shallow arc contour 46 as well as at the base 38 of the funnel body 40 of the device 16. The funnel body 40 is contoured while narrowing to the base 38 to provide some additional anchoring as the vaginal walls collapse. As described above a portion of the protruding lip 30 adjacent the thinner section also may be thinner than the rest of the protruding lip 30 to further facilitate folding of the fluid collection funnel device 16. In place of or in addition to the thinner section, the fluid collection funnel device 16 may also include a pressure point (as shown in FIGS. 14A and 15A) on the anterior surface of the fluid collection funnel device 16 adjacent the protruding lip 30. Each of the features described with regard to FIGS. 2-5, 14, and 15 may also be included in this example.

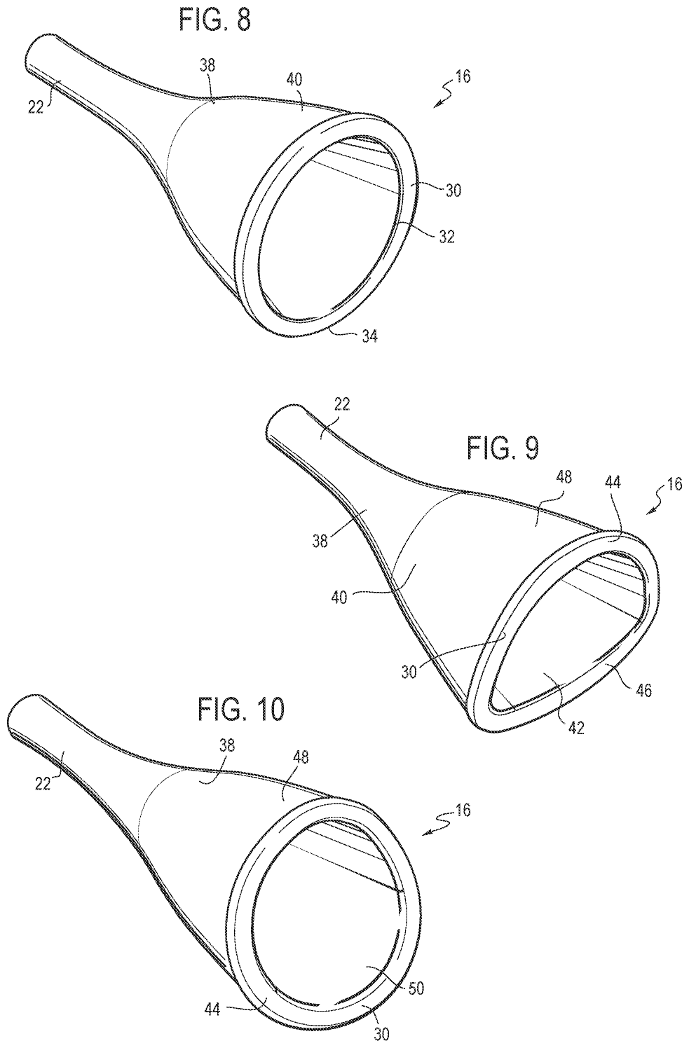

FIGS. 8-10 show the different example fluid collection funnels 16 of FIGS. 2-7 with shorter stems 22. As shown and more fully described above with reference to FIGS. 2-7, each fluid collection funnel device 16 has a funnel body 40, a protruding lip 30, and is configured and designed to be connected to drainage tubing 14, which in turn is connected to a fluid collection receptacle 12.

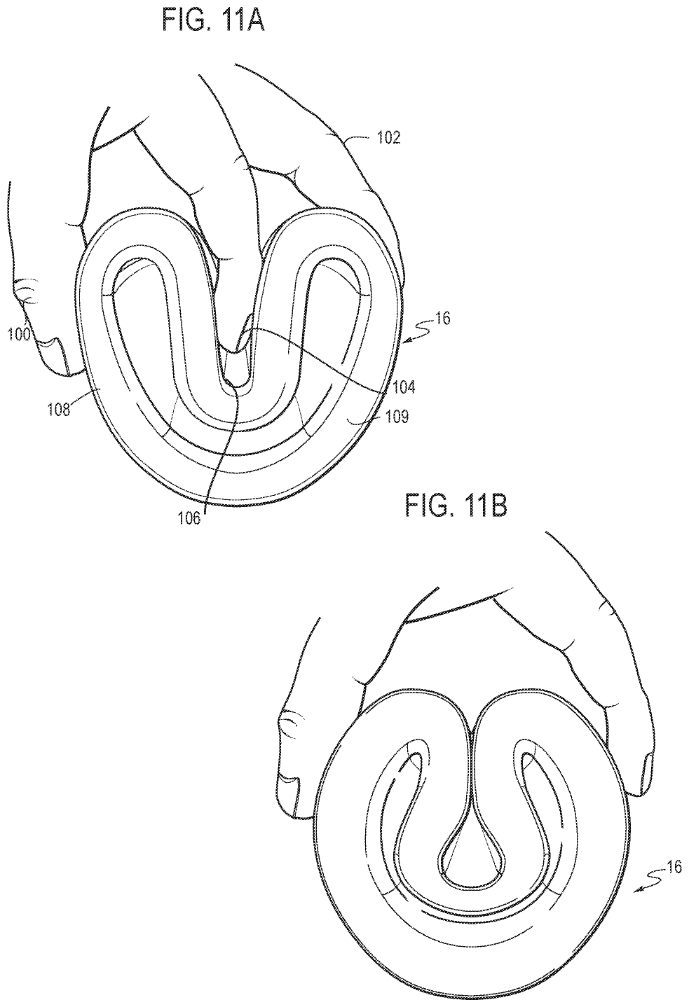

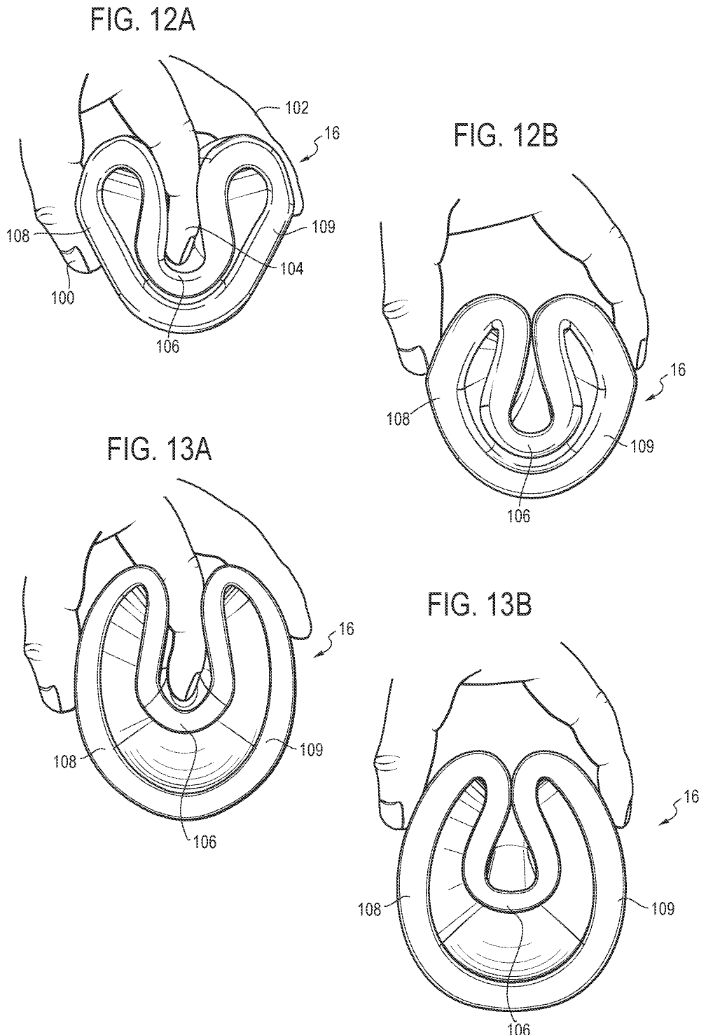

FIGS. 11A and 11B show views of a circular fluid collection funnel device 16 folded for insertion. FIGS. 12A and 12B show views of a semicircular fluid collection funnel device 16 folded for insertion. FIGS. 13A and 13B show views of a semicircular fluid collection funnel 16 having an extension folded for insertion. In each of FIGS. 11A, 12A, and 13A, a person grasps the fluid collection funnel 16 between their thumb 100 and their other fingers 102, while pressing their index finger 104 on the pressure point or region on the top/anterior 106 part of the device 16. As shown, the top or anterior part 106 of the funnel 16 is folded inwardly while the person holds adjacent sides 108, 109 of the device 16 with the thumb 100 and remaining fingers 102. As the index finger 104 is pulled back, the thumb 100 should be brought towards the other fingers 102 in a pinching motion, which forces the device 16 to collapse and be held in a folded position using as few as two fingers, as shown in FIGS. 11B, 12B and 13B. The lip 30 is constructed so that a technician can easily fold the funnel 16 for insertion and also rebound back to an unfolded state after released by the technician.

Once the funnel device 16 is folded, it can be placed within the vagina approximately halfway between the introitus and cervix. The precise location of anchoring may be determined by the individual physician during placement. Once anchoring location is determined, the device 16 is released and allowed to expand to its original shape. The funnel device 16 includes a protruding lip 30 that assists with anchoring the device 16. Once the funnel 16 is allowed to unfold within the vaginal canal, the protruding lip 30 engages the vaginal wall.

Immediately after the fluid collection funnel 16 is placed or prior to placement in the patient, the collection receptacle 12 is attached to the distal open end 25 of the stem 22 of the funnel 16 via the drainage tubing 14 (if not already attached). The distal open end 25 of the stem 22 accepts a conical connector at the proximal end 18 of the drainage tubing 14. If postpartum hemorrhage is occurring, the blood loss collection device 10 will funnel blood into the tubing 14 and subsequently into the collection receptacle 12, where total volume of blood loss can be monitored in real time. At 500 mL of blood loss, intervention to begin treatment of a hemorrhage can be considered and 1000 mL of blood loss is considered a critical threshold and requires response. Beyond this threshold of blood loss, the device 10 can be removed for access to the uterus for treatment. The device 10 can be removed from the vagina by inserting two fingers and folding the funnel device 16 before pulling it from the vagina.

FIGS. 14A-C show alternative examples of fluid collection funnels 200. FIG. 14A shows a fluid collection funnel 200 having a funnel body 202, a symmetrically circular lip 204, a symmetrically circular proximal opening 205, and a stem 206 extending from a base 208 of the funnel body 202. The funnel body 202 has an anterior surface 210 and a posterior surface 212 opposite the anterior surface 210. As shown in FIG. 14B, the anterior surface 210 tapers from the symmetrically circular lip 204 to the base 208 of the funnel body 202. An opening 213 shown in FIG. 14C is provided in the base 208 of the fluid collection funnel 200, which leads into stem 206. The distal end of the stem 206 has a distal opening 216. Returning to FIG. 14B, the anterior surface 210 has a continuous and contoured taper extending from the lip 204 to the base 208. The stem 206 extending from the base 208 has a slight flare 214 at its end 216 to receive a connector of the tubing 14.

The posterior surface 212 extending from the lip 204 to the base 208 has little or no taper, thereby providing a flattened or laminar flow path within the interior of the funnel 200 from the symmetrically circular opening 205 to and through the stem 206 to prevent pooling of blood in the funnel body 202 of the funnel 200 when the patient is lying supine or semi-supine after giving birth, thereby preventing inaccuracies in hemorrhaging measurement. The lip 204 protrudes uniformly from the exterior wall 211 at the opening 205, but does not extend into the interior of the funnel 200 thereby avoiding any obstruction or impeding of fluid flow. The interior surface of the funnel presents a smooth and uniform surface free of any protrusions or other obstruction.

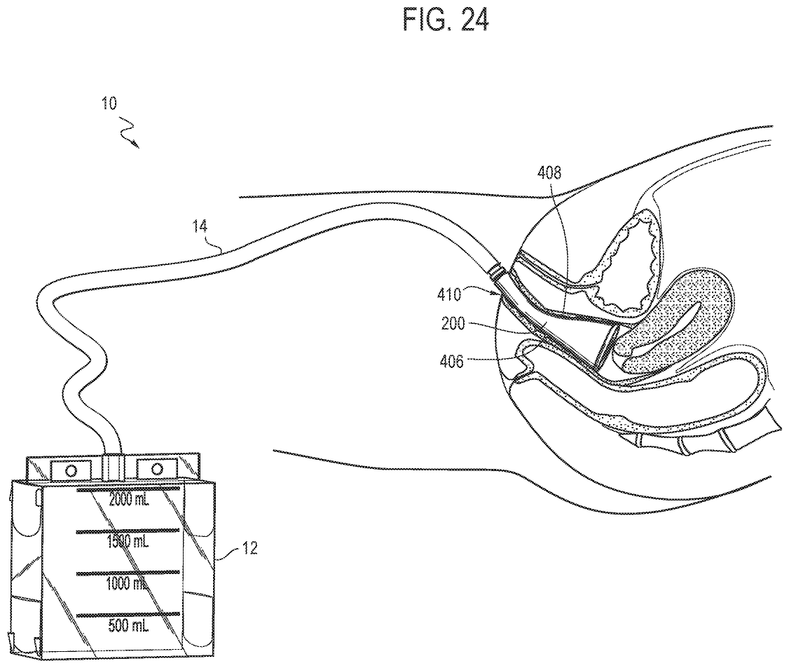

As shown in FIGS. 14A-B and 15A-B, the posterior surface 212 has little or no taper from the lip 204 to the base 208 and therefore no bulge in which blood can pool in the interior. The stem 206 extends from the base 208 and has a slight flare 214 toward its open distal end 216 to facilitate attachment to a connector (not shown), for example a barb connector, of a drainage tube, such as the drainage tube 14 of FIG. 1. As shown in FIGS. 14A-C, the stem 206 of the funnel 200 is substantially flush with the posterior surface 212 of the funnel body 202 and is offset from the geometric center of the funnel 200 to provide the flattened flow path in the interior of the funnel 200. FIG. 14C shows a front view of the funnel 200, and the interior of the funnel 200, having an internal anterior surface 210′ and an interior posterior surface 212′. As shown in FIG. 14B, the stem 206, except for the slight taper 214 at the distal end 216, is substantially flush with the posterior surface 212. Hence, the interior posterior surface 212′ of the funnel body 202 is substantially flush with the interior posterior surface 212′ of the stem 206 such that fluid flow from the opening 205 to and through the open distal end 216 is flat and laminar. The interior posterior surface 212′ has little, or in some examples no, taper from the opening 205 to and through the base 208. This shape of the fluid collection funnel 200 is designed and configured such that when the funnel 200 is inserted into a patient, as shown in FIG. 24, the anterior surface 210 of the funnel 200 engages and conforms to the anterior vaginal wall 218 and the posterior surface 212 engages and conforms to the posterior vaginal wall 220 and fluid is prevented from pooling in the funnel 200.

In the examples of FIGS. 14A-C and 15A-B, the fluid collection funnel 200 has a pressure point 201 or section adjacent the lip 204 of the funnel 200 and extending from the lip 204. In some examples, the pressure point 201 is directly adjacent the lip 204. The pressure point 201 or section has, as described above, a lesser thickness of material compared to other portions of the funnel 200. Additionally or optionally, the pressure point 201 has a lower durometer than other portions of the funnel 200 to facilitate folding, or may be a different, more pliable material to facilitate folding. As shown in FIGS. 14A and 15A, a portion 232 of the lip 204 of the funnel 200 at the top of the funnel adjacent the pressure or folding section 201 is thinner than other portions of the lip 204, which also facilitates folding. While the lip 204 in this region may be thinner than the rest of the lip 204, the lip 204 and the opening 205 both remain symmetrically circular in these examples. As shown in FIGS. 15A and B, the funnel body 202 may include grips 234, such as, for example, ridges, dimples, grooves, etc., on an outer surface to facilitate removal of the funnel 200. Ridges or other gripping features may be provided on and along the stem 206, as well, to facilitate removal.

FIGS. 16A and B shown a further example of a fluid collection funnel device 200. In this example, the fluid collection funnel 200 has similar features to the funnel described with regard to FIGS. 14 and 15 above, but includes a pre-curved end 209 at the base 208 to conform to a patient's anatomy. As shown, the posterior surface 212 (as well as the interior posterior surface 212′) has no taper and provides a flattened, unobstructed flow path from the opening 205 to the base 208.

The fluid collection funnels 200 of FIGS. 14A-16B may be constructed from soft, medical-grade silicone and is free from phthalate, latex, dioxin, & BPA. The durometer may range from 40A to 70A and, in some examples, is from about 50A to 60A. The thinner section and/or pressure point or region may be of a lower durometer and in the range of about 20A to 40A.

With each of the examples of FIGS. 1-16B, the funnel 16, 200 is shaped and configured to enter the anatomy following birth and anchor within the vagina. The protruding lip 30, 204 of each funnel 16, 200 creates a firm anchoring feature with which the insertable portion of the device can press against the vaginal walls. The interior-most edge of the lip 30, 204 may not be concentric with the exterior most edge and may slope inwardly or outwardly, as described above. The funnel 200 of the examples shown in FIGS. 14A-15B may have a thinner section gradually thickening both circumferentially and axially along the insertable portion of the device and achieving maximum thickness 90° circumferentially from the thinner section and at the base of the funnel body of the device. As described above, the funnel body contoured on its anterior surface while narrowing to the base to provide some additional anchoring as the vaginal walls collapse. Each of the features described with regard to FIGS. 2-10 may be included in the examples of FIGS. 14A-16B.

The fluid collection funnel 16, 200 as described herein may have a dimension in a range of about 3 to about 6 inches in length from the opening to the distal end of the stem, and in some examples, of about 4 to about 5 inches in length from the opening to the distal end of the stem. The inner diameter of fluid collection funnel at the opening has a dimension in a range of about 1.5 to about 2.5 inches, and in some examples, of about 1.25 to about 2.25 inches. The thickness of the lip from top to bottom is in a range of about 0.1 inches to about 0.2 inches, and in some examples, of about 0.13 to about 0.17 inches, and in some examples, about 0.15 inches. The outer diameter of the fluid collection funnel at the opening has a dimension in a range of about 0.50 to about 4.0 inches, and in some examples, of about 0.65 to about 3.75 inches, and in some examples, of about 0.8 to about 3.5 inches, and in some examples, of about 1.0 to about 3.25 inches, and in some examples, of about 1.15 to about 3.0 inches, and in some examples, of about 1.3 to about 2.75 inches, and in some examples, of about 1.45 to about 2.5 inches, and in some examples, of about 1.6 to about 2.25 inches, and in some examples, of about 1.7 to about 2.0 inches. The angle of the taper at the distal opening of the stem has a dimension in a range of about 4.5° to about 6.5°, and in some examples, of about 5° to about 6°. The inner diameter of the base of the fluid collection funnel has a dimension in a range of about 0.2 to about 0.35 inches, and in some examples, of about 0.25 to about 0.3 inches. The radius of the lip has a dimension in a range of about 0.05 inches to about 0.10 inches, and in some examples, of 0.07 to about 0.08 inches, and in some examples, about 0.075 inches. The dimensions are an approximate of the female human anatomy to provide the most effective and comfortable fit for the patient and for accurate fluid loss measurement.

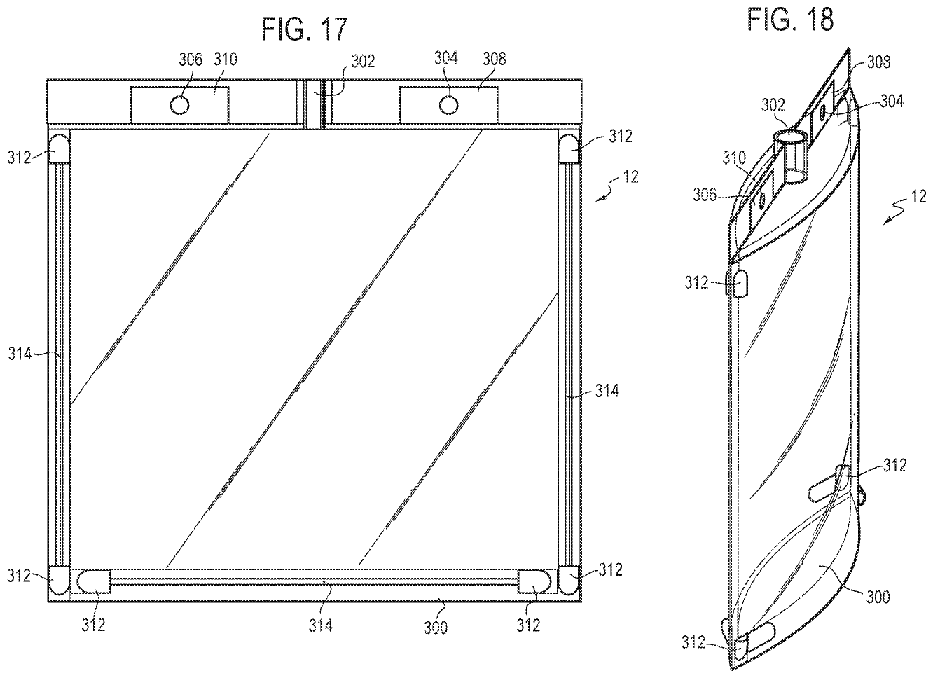

FIGS. 17 to 22 show various fluid collection receptacles that are designed and configured to be attached via a drainage tube to the drainage end of a fluid collection funnel. The collection vessel is configured to collect postpartum blood and to provide a reliable and accurate measurement of this volume, allowing the user or a technician to clearly identify when the critical blood loss threshold has been reached. The device may be easy and quick to implement, intuitive to use in an emergency, and may have a low training burden. Additionally, the device may be cost effective and should have packaging that takes up minimal space.

Common fluid collection bags on the market experience issues such as wall adhesion and ballooning, which contribute to inaccuracies in volume readings. Such flat bags with two layers of film sealed along the perimeter take the 3D shape of the fluid collected. This design also allows the film layers to adhere to one another during the filling process, contributing to the inaccuracy. These drainage bags often have hooks or straps to secure the bag to a structure in the use environment, such as hanging the bag on a hospital bed. The fluid collection receptacles of the present disclosure are designed and configured for use in an environment where a structure to hang the collection vessel from are unavailable. Thus, the collection vessel self-standing and can provide accurate volume measurements.

While a rigid container stands on its own and maintains its shape while accurately measuring volume, a 2000 ml bottle would take up significant packaging space and are potentially more expensive than a foldable and expandable collection receptacle.

The present disclosure provides examples of fluid collection receptacles that can have a 3D shape in use like a rigid bottle, but are foldable for packaging, storage, and can easily and quickly deploy from its foldable state. The receptacles 12 described herein are designed and configured to be free-standing, collapsible (and collapsed in one configuration) and expandable in the in use configuration. The receptacles 12 may be self-expanding, where they spring open upon release from compression, or physically movable into the expanded configuration.

A fluid collection receptacle 12 is shown in FIGS. 17 and 18. One example of the collection receptacle 12 as part of the blood loss collection device 10 is shown in FIG. 1. As shown in FIG. 18, the receptacle 12 features a semi-cylindrical profile with a flat base 300 that allows the receptacle 12 to stand upright on a flat surface. FIG. 17 shows the receptacle 12 in its flattened configuration. Tubing 14 connects (as shown in FIG. 1) to the receptacle 12 at a cylindrical inlet port 302. An outer diameter of the tubing 14 mates with an inner diameter of the inlet port 302. Two holes 304, 306 are located at the top of the receptacle 12 and can be used for attaching the receptacle 12 to structures in the use environment, if available. The holes 304, 306 and base 300 allow the receptacle 12 to be both hangable as well as free-standing on a surface. The rectangular features 308, 310 surrounding these holes 304, 306 reinforce and strengthen the receptacle material at these regions. These features accommodate the increased weight of the collected blood and prevent receptacle 12 from separation from the attachments.

Pocket features 312 are provided in the corners of the front and back faces of the receptacle 12. These pockets 312 constrain flexible, insertable rod-like components 314 that conform to and support the receptacle profile in the vertical and horizontal dimensions. These metal or plastic rod-like components 314 provide structure to the receptacle 12 and help retain the receptacle's 3D shape while collecting blood to ensure accurate volume measurements. Such rod components 314 could also be constrained by sleeves or channels of film material instead of the pockets 312. The flexibility of the horizontal rods 314 ensures that the receptacle base 300 remains expanded, creating the receptacle's semi-cylindrical profile. This flexibility also allows the receptacle base 300 and the front and back faces to compress in packaging to minimize occupied storage space. When the receptacle 12 is removed from packaging, the compressed rod 314 will spring back to its constrained form, which allows the receptacle 12 to pop open into its semi-cylindrical profile. Other specific design features such as the receptacle film material and thickness minimize adhesion and ballooning of the receptacle walls to promote accurate measurements. Thermoplastic polyurethane film materials have been investigated as environmentally sustainable alternatives to vinyl. Example materials for the support rods 314 include acrylic, HDPE, LDPE, PVC, PETG, PP, structural FRP fiberglass, stainless steel, Nitinol and aluminum. For example, the rods 314 may be made of super elastic and/or shape memory material which upon release from compression remember a pre-set shape, allowing the receptacle to spring open.

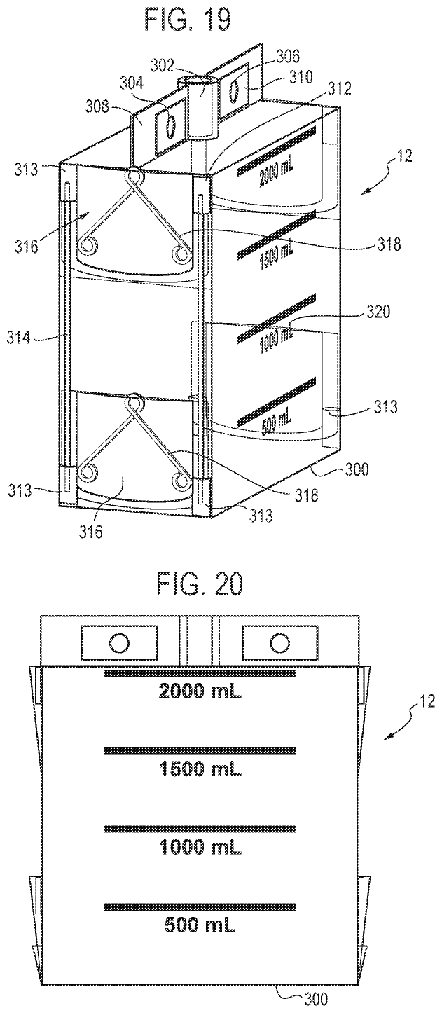

FIGS. 19 and 20 show an alternative example of a rectangular collection receptacle 12. Here, the collection receptacle 12 has a rectangular configuration. This bag 12 features a rectangular profile with a flat base 300 that allows the receptacle to stand upright on a surface in the use environment. Tubing 14, as shown in FIG. 1, connects to the receptacle 12 at the cylindrical inlet port 302. The tubing outer diameter mates with the inner diameter of the inlet port 302. Two holes 304, 306 are located at the top of the receptacle 12 for attachment to a hook or stand. The rectangular features 308, 310 surrounding these holes 304, 306 reinforce and strengthen the receptacle material at these regions, ensuring that the receptacle 12 does not separate from its attachment due to the weight of added blood. Pocket features 313 are present on the sides of the receptacle 12. The pockets 313 constrain flexible, insertable components 314 that conform to the receptacle profile to retain the receptacle's vertical dimensions. These metal or plastic rod-like components provide structure to the receptacle 12 and help it to retain its 3D shape and flat base 300 while collecting blood to promote accurate volume measurements.

Larger pockets 316 constrain torsional spring components 318 that push the front and back receptacle faces away from one another, which help to retain the width dimensions. With these spring 318s, the front and back receptacle faces can be compressed together to minimize packaging size. When the receptacle 12 is removed from packaging, the expanding torsion spring 318 will push the receptacle faces away from one another, causing the receptacle 12 to assume its expanded, rectangular profile. A sample design of graduation markings 320 is displayed on the front face of the rectangular receptacle 12. These markings at 500 ml increments provide visual indication when critical PPH volume thresholds have been reached.

In some examples, the rod components 314 could be constrained by sleeves or channels of film material instead of by the pockets shown in FIGS. 17 and 18. The inlet port 302 could be positioned on the back face of the receptacle 12 opposite the front face with the volume markings 320 to aid with receptacle folding and for improved visualization of the printed graduations. If tubing enters an inlet port 302 positioned on the back face, the inlet port 302 at the top of the receptacle in FIG. 18 could be replaced with additional hanging reinforcement. A membrane/filter disk (not shown) as is known in the art could be added to the receptacle 12 for air evacuation and consistent fluid filling.

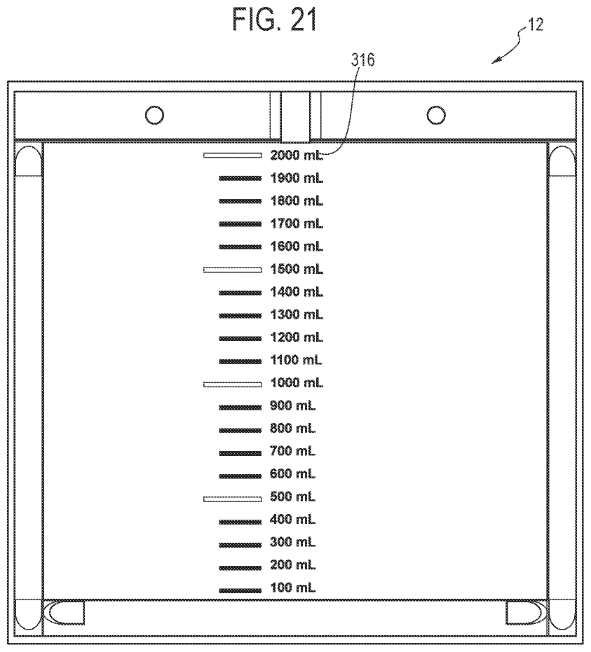

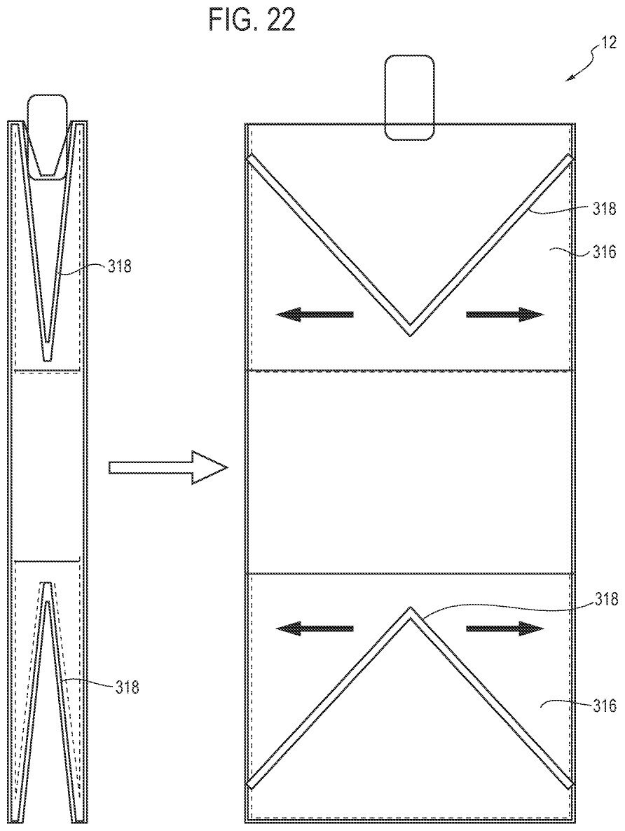

FIG. 21 shows a sample design of graduation markings 320 for the receptacles. This includes 100 ml increments up to 2000 ml along with adjacent lines. The lines at every 500 ml increment are larger in size, providing visual emphasis when critical PPH volume thresholds have been reached.

FIG. 22 shows the rectangular receptacle's side profile when folded in packaging and when deployed. The receptacle's base 300 folds in half upon compression of the front and back faces and the torsion springs 318. The torsion springs 318 are depicted as the triangular structures with an alternative design profile compared to those shown in FIG. 19. The pockets 316 constraining the torsion springs 318 are indicated by the rectangles 316. When the receptacle 12 is removed from packaging, the torsion springs 318 expand the sides, and the receptacle 12 assumes its expanded rectangular profile. Alternatively, the receptacle 12 could be delivered with a protective sleeve or clip mechanism retaining the receptacle 12 in its folded state in and out of the packaging. This format could allow the health care professional to deploy the receptacle 12 at the appropriate time by removing the sleeve or clip. In a third deployment example, the rods 314 and springs 318 could be included as separate components with the folded receptacle in packaging. After removing the receptacle 12 from packaging and unfolding, the user could insert the included rods 314 and springs 318 to hold the receptacle 12 open, establishing a defined 3D rectangular profile. This contrasts with previously discussed examples where the rods 314 and springs 318 are already incorporated into sleeves/channels/pockets on the receptacle 12 and do not require assembly by the user.

Example materials for the torsion springs 318 include spring steel, Nitinol, or plastics such as those listed for the support rods 314.

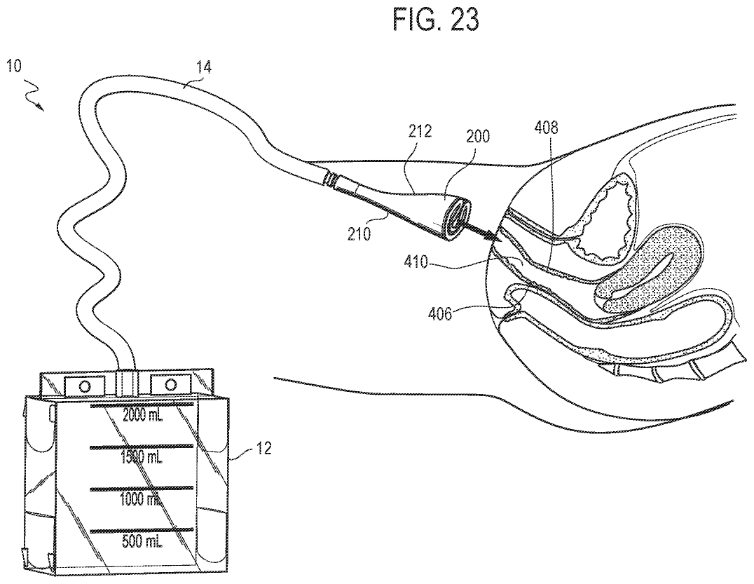

FIG. 23 shows patient anatomy and a system 10 for monitoring post-partum hemorrhaging in the patient prior to insertion of a fluid collection funnel 200 and FIG. 24 shows patient anatomy and a system 10 for monitoring post-partum hemorrhaging in the patient after insertion of a fluid collection funnel 200. As shown in FIG. 23, the fluid collection funnel 200 is connected by drainage tubing 14 to the fluid collection receptacle 12. The funnel 200 is shown in the compressed configuration in FIG. 23 prior to insertion into the patient anatomy. As shown in FIG. 24, the funnel 200 is inserted in place into the vaginal canal 410. The contoured anterior surface 212 of the funnel 200 is disposed against an anterior wall 408 of the vagina 410 and a flattened posterior surface 210 is disposed against a posterior 406 of the vagina 410. When in place, the internal posterior surface 212′ of the funnel 200 presents an unobstructed flow path.

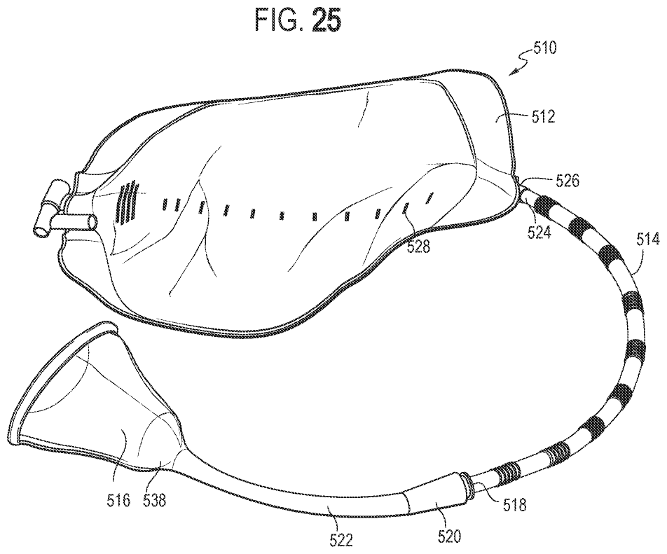

FIG. 25 shows a different example blood loss collection system 510. The system 510 includes an expandable fluid collection receptacle 512, drainage tubing 514, and fluid collection funnel 516. A first end 518 of the fluid connection end of the tubing 514 is sealingly connected to a drainage end 520 of a stem 522 of the fluid collection funnel 516. A second end 524 of the drainage tubing is sealingly connected to a port 526 of the collection receptacle 512. The collection receptacle 512 includes markings 528 to indicate the collected volume of blood. The drainage tubing 514 extends from the collection receptacle 512 to the stem 522 of the fluid connection funnel 516. The stem 522 of the funnel 516 connects to the first end 518 of the tubing 514 by friction-fit, and in some cases, by a conical connector.

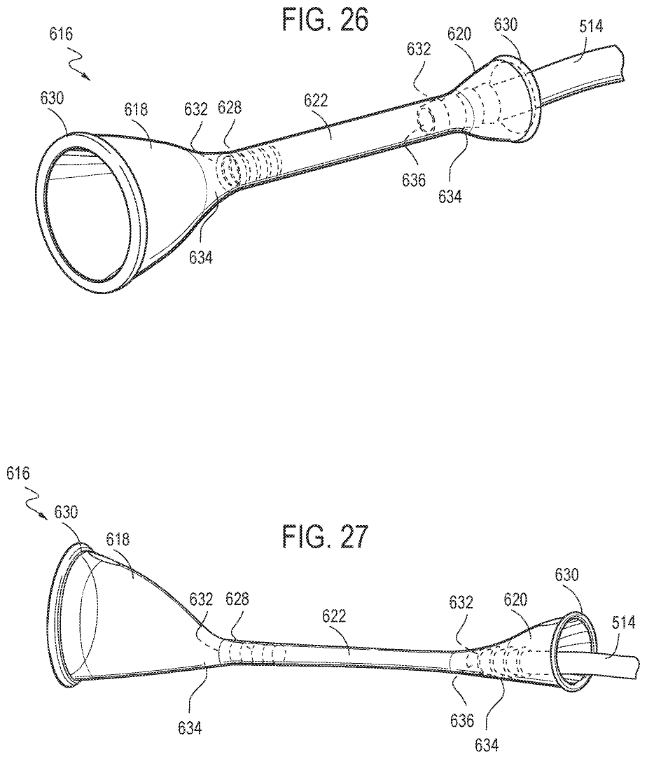

Variations of the fluid collection device are shown in FIGS. 26 and 27. In each of FIGS. 26 and 27, the device 616 is a double funnel device having a funnel 618, 620 at either end of a central tube 622. The device 616 of FIG. 26 may be similarly constructed to the cup of FIG. 8, and the device 616 of FIG. 27 may be similarly constructed to the cup of FIGS. 14A-C. As shown in FIG. 26, the double funnel device 616 has a first funnel 618 at one end and a second funnel 620 at the other end. As shown, the first funnel 618 is larger than the second funnel 620. Tubing 622 extends between the first and second funnels 618, 620 and has a lumen in fluid communication with each of the funnels 618, 620. Connectors 628 are provided on an interior surface of the tubing 622 to secure a drainage tubing 610 to the device 616. Specifically, the connector 628 is formed within the tubing 622 adjacent an opening 632 at a base 634 of both of the funnels 618, 620. The opening 632 of each funnel 618, 620 may have the same or different diameters from the other funnel 618, 620.

As shown in FIGS. 26 and 27, drainage tubing 514, which is connected to the second funnel 620, has a corresponding connector 636 at its end. The drainage tube 514 is configured for decoupling from the second funnel 620 and coupled to the first funnel 618 at the connector 628. Each of the funnels 618, 620 has a rim 630 configured to engage with and seal against the vaginal canal. Other constructions of the double-funnel device may be similar to any of the cup constructions described herein.

While various examples of the disclosure have been described, the disclosure is not to be restricted except in light of the attached claims and their equivalents. Moreover, the advantages described herein are not necessarily the only advantages of the disclosure and it is not necessarily expected that every example of the disclosure will achieve all of the advantages described. Each of the examples described further may include all or only a subset of the features described herein.

What is claimed is:

1. A fluid collection device of a postpartum hemorrhage monitoring system, the device comprising:
a funnel body comprising:
an open proximal end,
an interior configured for receiving a fluid,
a rim extending exteriorly from the open proximal end,
an anterior outer surface configured for placement adjacent an anterior vaginal wall of a patient,
a posterior outer surface configured for placement adjacent a posterior vaginal wall of the patient, and
an open distal end; and
a stem having an open proximal stem end, an open distal stem end, and a lumen extending therebetween, the stem extending from the open distal end of the funnel body and configured for receiving fluid from the interior of the funnel body;
wherein the anterior surface tapers to the proximal stem end and the posterior outer surface is flush with the stem such that the stem is rearward of a geometric center of the funnel body to facilitate movement of fluid into and through the stem and minimize accumulation of fluid within the funnel body when the patient is in a supine position.

2. The device of claim 1, wherein the stem is entirely rearward of the geometric center of the funnel body.

3. The device of claim 1, comprising a pressure point disposed adjacent the rim to facilitate folding of the rim and the funnel body for placement within a patient.

4. The device of claim 1, comprising a pressure point to facilitate folding of the rim and the funnel body for placement within a patient, wherein the pressure point is disposed in an area of the funnel body having a durometer less than a durometer of a remainder of the funnel body.

5. The device of claim 1, comprising a pressure point to facilitate folding of the rim and the funnel body for placement within a patient, wherein the pressure point is an area of the funnel body that is thinner than a remainder of the funnel body.

6. The device of claim 4, wherein the pressure point is disposed adjacent the rim.

7. The device of claim 4, wherein the pressure point comprises an indentation.

8. The device of claim 1, comprising a second funnel body extending from the open distal stem end.

9. The device of claim 8, wherein the second funnel body is smaller than the funnel body.

10. A fluid collection device for a postpartum hemorrhage monitoring system, the device comprising:
a funnel body comprising:
an open proximal end,
an interior configured to receive postpartum hemorrhage,
a symmetrically circular rim extending exteriorly from the circular open proximal,
an anterior outer surface configured for placement adjacent an anterior vaginal wall of a patient,
a posterior outer surface configured for placement adjacent a posterior vaginal wall of a patient,
an open distal end,
a pressure point to facilitate folding the funnel for placement with a patient,
a stem having an open proximal stem end, an open distal stem end, and a lumen extending therebetween, the stem extending from the open distal end of the funnel body and configured for receiving fluid from the interior of the funnel body, and
wherein the anterior outer surface tapers toward the proximal stem end, the posterior outer surface being substantially flush with the stem, and the stem being offset from a geometric center of the funnel body to facilitate movement of fluid into and through the stem and minimize accumulation of fluid within the funnel body when the patient is in a supine position.

11. The device of claim 10, wherein the stem is entirely rearward of the geometric center of the funnel body.

12. The device of claim 10, wherein the pressure point is disposed adjacent the rim.

13. The device of claim 10, wherein the pressure point is disposed in an area of the funnel body having a durometer less than a durometer of a remainder of the funnel body.

14. The device of claim 10, wherein the pressure point is an area of the funnel body that is thinner than a remainder of the funnel body.

15. A postpartum hemorrhage monitoring system comprising:
a drainage tube;
a collapsible collection bag configured for coupling to the drainage tube;
a fluid collection funnel configured for coupling to the drainage tube, the fluid collection funnel comprising:
an open proximal end,
an interior configured to receive postpartum hemorrhage,
a rim extending exteriorly from the open proximal end,
an anterior outer surface configured for placement adjacent an anterior vaginal wall of a patient,
a posterior outer surface configured for placement adjacent a posterior vaginal wall of a patient,
an open distal end;
a stem having an open proximal stem end, an open distal stem end, and a lumen extending therebetween, the stem extending from the open distal end of the fluid collection funnel and configured for receiving fluid from the interior of the fluid collection funnel; and
a pressure point disposed adjacent the rim to facilitate folding of the rim and the fluid collection funnel for placement within a patient,
wherein the anterior surface tapers to the proximal stem end and the posterior surface is flush with the stem such that the stem is substantially rearward of a geometric center of the fluid collection funnel to facilitate movement of fluid into and through the stem and minimize accumulation of the fluid within the fluid collection funnel when the patient is in a supine position.

16. The system of claim 15, wherein the drainage tube is configured for coupling to the open proximal end of the stem, and the collapsible collection bag is configured for receiving fluid from the fluid collection funnel by way of the drainage tube.

17. The system of claim 15, wherein the collapsible collection bag has a collapsed configuration and an expanded configuration.

18. The system of claim 17, wherein in the expanded configuration, the collapsible collection bag is freestanding.

19. The system of claim 18, wherein the collapsible collection bag is self-expanding upon release from the collapsed configuration.

20. The system of claim 15, further comprising a drainage tube and a self-expandable fluid collection bag configured to be free-standing and self-supporting upon expansion.
